(19) United States
(12) Patent Application Publication
(10) Pub. No.: US 2018/0306662 A1
(43) Pub. Date: Oct. 25, 2018

(54) System and Methods for Chromatic Pressure Gauges

(71) Applicants: ADAM BURCHETT, BREMERTON, WA (US); DONALD CONNER LEGARE, BREMERTON, WA (US); ALEX CORDOVA, PORT ORCHARD, WA (US); DAIN COX, POULSBO, WA (US); BRYANT VEACH, SILVERDALE, WA (US)

(72) Inventors: ADAM BURCHETT, BREMERTON, WA (US); DONALD CONNER LEGARE, BREMERTON, WA (US); ALEX CORDOVA, PORT ORCHARD, WA (US); DAIN COX, POULSBO, WA (US); BRYANT VEACH, SILVERDALE, WA (US)

(21) Appl. No.: 15/961,788

(22) Filed: Apr. 24, 2018

Related U.S. Application Data
(60) Provisional application No. 62/489,071, filed on Apr. 24, 2017.

(51) Int. Cl. G01L 11/02

(52) U.S. Cl. CPC G01L 11/02; A61F 9/029

(57) ABSTRACT

Disclosed herein are systems for quick and convenient indication of pressure acting upon a vessel. Such systems are based on the color of a chromatic material at a corresponding pressure. Systems include a real-time and optical system for gauging pressure that allows users to make safe judgements or determine the state of a vessel. The color of the mechanochromic material varies as the material deforms and provides users with a sense of the pressure within a vessel. The color of the piezochromic material varies as the fluid pressure changes. Systems include an integrative and adaptive chromatic material that may be applied across a range of pressurized vessels.

### COPYRIGHT NOTICE

Contained herein is material that is subject to copyright protection. The copyright owner has no objection to the facsimile reproduction by anyone of the patent document or the patent disclosure, as it appears in the United States Patent and Trademark Office patent file or records, but otherwise reserves all rights to the copyright whatsoever. The following notice applies to the software, screenshots and data as described below and in the drawings hereto and All Rights Reserved.

### TECHNICAL FIELD

This disclosure relates generally to pressure measurement in a variety of applications. More specifically, it focuses on the implementation of mechanochromic and piezochromic materials in pressure vessels as a way of measuring the pressure within a vessel.

### BACKGROUND

Many devices and systems depend on an internal pressure that is different from the surrounding pressure to function properly. This difference in pressure can be positive or negative pressure. The pressure of a vessel is largely responsible for providing useful properties such as mechanical shock absorption in car tires or high coefficient of restitution in sports balls.

Pressure is most commonly measured using mechanical or digital pressure gauges. Mechanical gauges are usually equipped with a rotating dial that aligns with calibrated markings of pressure. Perhaps the most common example of a mechanical pressure gauge is the Bourdon-tube gauge. Digital pressure gauges rely on piezoresistive or piezoelectric properties of internal components to display pressure as a number on an electronic display. Digital pressure gauges are ideal for small incremental pressure changes where an analog gauge may not provide the required precision.

Both types of gauges are suitable for a variety of applications, but they also have shortcomings. Mechanical and digital gauges tend to be complex with many small internal parts, making them expensive to manufacture and purchase. The small parts located within the gauges are also prone to malfunction or breaking if they are not handled with care. In addition, many applications, such as sports balls, require a needle and hose attachment to measure the internal pressure. This makes even the smallest or most portables gauges inconvenient to carry and operate. Existing gauges are also impractical for use in time-sensitive scenarios. For example, a sports game cannot afford a pause in the action to check the pressure of the ball. Doing so would result in a loss of interest from people watching.

What is needed is a quick, convenient, and easy-to-use pressure gauge that has the versatility and durability to be incorporated directly into many different pressurized systems. A pressure gauge with these qualities would allow users to determine pressure with a speed and level of simplicity that isn't possible with existing pressure gauges.

So as to reduce the complexity and length of the Detailed Specification, Applicant(s) herein expressly incorporate(s) by reference all of the following materials identified in each paragraph below. The incorporated materials are not necessarily “prior art” and Applicant(s) expressly reserve(s) the right to swear behind any of the incorporated materials.

Ingestibles Possessing Intrinsic Color Change, U.S. Pat. No. 6,866,863 filed Jun. 23, 2000 is herein incorporated by reference in its entirety.

Self-Assessing Mechanochromic Materials, U.S. Pat. No. 8,236,914 filed Jan. 26, 2010, is herein incorporated by reference in its entirety.

Coating Composition Having Mechanochromic Crystals, U.S. Pat. No. 913,336, filed Jul. 16, 2012 is herein incorporated by reference in its entirety.

Mechanochromic Coating Composition, U.S. Pat. No. 8,815,771, filed Apr. 16, 2012 is herein incorporated by reference in its entirety.

Toothbrush, U.S. Pat. No. 6,330,730, filed Aug. 1, 1997 is herein incorporated by reference in its entirety.

Compounds for reducing background color in color change compositions, U.S. Pat. No. 9,528,004, filed Mar. 14, 2014 is herein incorporated by reference in its entirety

Applicant(s) believe(s) that the material incorporated above is “non-essential” in accordance with 37 CFR 1.57, because it is referred to for purposes of indicating the background or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

Aspects and applications presented here are described below in the drawings and detailed description. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112, ¶ 6. Thus, the use of the words “function,” “means” or “step” in the Detailed Description or Description of the Drawings or claims is not intended to somehow indicate a desire to invoke the special provisions of 35 U.S.C. § 112, ¶ 6, to define the systems, methods, processes, and/or apparatuses disclosed herein. To the contrary, if the provisions of 35 U.S.C. § 112, ¶ 6 are sought to be invoked to define the embodiments, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ”, if the claims also recite any structure, material or acts in support of that means or step, or that perform the recited function, then it is the clear intention of the inventors not to invoke the provisions of 35 U.S.C. § 112, ¶ 6. Moreover, even if the provisions of 35 U.S.C. § 112, ¶ 6 are invoked to define the claimed embodiments, it is intended that the embodiments not be limited only to the specific structure, material or acts that are described in the preferred embodiments, but in addition, include any and all structures, materials or acts that perform the claimed function as described in alternative embodiments or forms, or that are well known present or later-developed, equivalent structures, material or acts for performing the claimed function.

### BRIEF DESCRIPTION OF THE DRAWINGS

A more complete understanding of the systems, methods, processes, and/or apparatuses disclosed herein may be derived by referring to the detailed description when considered in connection with the following illustrative figures. In the figures, like-reference numbers refer to like-elements or acts throughout the figures.

FIG. 1A depicts a mechanochromic membrane under the skin of a vessel where it may be viewed through an aperture, where the internal pressure is greater than the external pressure.

FIG. 1B depicts FIG. 1A working in reverse where the external pressure is greater than the internal pressure.

FIG. 2A depicts an embodiment of a valve with a mechanochromic membrane and a window.

FIG. 2B depicts a portion of the valve embodiment of FIG. 2A.

FIG. 2C depicts the valve embodiment of FIG. 2B including a change in internal pressure.

FIG. 3A depicts an embodiment of a valve comprising a piezochromic layer.

FIG. 3B depicts the valve embodiment of FIG. 3A where a compressible fluid separates a piezochromic layer from the window.

FIG. 3C depicts the valve embodiment of FIG. 3B with an increase in pressure affecting the piezochromic layer.

FIG. 4A depicts a cross sectional view of an inflation orifice where a pressurized mechanochromic vessel is used to indicate pressure.

FIG. 4B depicts a pressurized mechanochromic indicator located within the nozzle of the vessel while the internal pressure is different from the external pressure.

FIG. 5 depicts a pressurized vessel embodiment comprising an external pressure, an internal pressure, a bladder, an exterior skin, and a valve.

FIG. 6A depicts a pressurized vessel with an exterior nozzle.

FIG. 6B depicts the exterior nozzle of FIG. 6A comprising a mechanochromic membrane and a window.

FIG. 6C depicts the exterior nozzle of FIG. 6A comprising a mechanochromic layer.

FIG. 6D depicts the exterior nozzle of FIG. 6A comprising a piezochromic layer.

FIG. 7A depicts a pressurized vessel comprising an input nozzle.

FIG. 7B depicts a section view of the input nozzle of FIG. 7A comprising a mechanochromic indicator around the base.

FIG. 7C depicts a cross sectional view of the exterior nozzle with the mechanochromic material visible.

FIG. 8A depicts a spacecraft where a mechanochromic indicator is used.

FIG. 8B depicts an embodiment of a mechanochromic indicator for use on a space suit.

FIG. 9 depicts an embodiment of a mechanochromic indicator for use on a container.

FIG. 10A depicts an embodiment of a mechanochromic indicator for use on a lid of a container.

FIG. 10B depicts a magnification of the lid of the container in FIG. 10A.

FIG. 11A depicts a scuba diver using chromatic indicators.

FIG. 11B depicts the air tank with a chromatic indicator used by the scuba diver in FIG. 11A.

FIG. 11C depicts a magnification of the chromatic indicator on the air line in FIG. 11A.

FIG. 12A depicts a construction device that uses hydraulic cylinders to lift loads.

FIG. 12B depicts a hydraulic piston where a mechanochromic material is applied to the outside of the piston cylinder.

FIG. 12C depicts the FIG. 13A where the piston cylinder has deformed.

FIG. 13A depicts an airplane with internal chromatic indicators.

FIG. 13B depicts a magnification of the airplane window in FIG. 14A with an external chromatic indicator.

Elements and acts in the figures are illustrated for simplicity and have not necessarily been rendered according to any particular sequence or embodiment.

### DETAILED DESCRIPTION

In the following description, and for the purposes of explanation, numerous specific details, process durations, and/or specific formula values are set forth in order to provide a thorough understanding of the various aspects of exemplary embodiments. It will be understood, however, by those skilled in the relevant arts, that the apparatus, systems, and methods herein may be practiced without these specific details, process durations, and/or specific formula values. It is to be understood that other embodiments may be utilized and structural and functional changes may be made without departing from the scope of the apparatus, systems, and methods herein. In other instances, known structures and devices are shown or discussed more generally in order to avoid obscuring the exemplary embodiments. In many cases, a description of the operation is sufficient to enable one to implement the various forms, particularly when the operation is to be implemented in software. It should be noted that there are many different and alternative configurations, devices, and technologies to which the disclosed embodiments may be applied. The full scope of the embodiments is not limited to the examples that are described below.

In the following examples of the illustrated embodiments, references are made to the accompanying drawings which form a part hereof, and in which is shown by way of illustration various embodiments in which the systems, methods, processes, and/or apparatuses disclosed herein may be practiced. It is to be understood that other embodiments may be utilized and structural and functional changes may be made without departing from the scope.

Disclosed herein are systems and methods for measuring the internal pressure of pressurized vessels using chromatic materials. For the purpose of discussion, materials that have special characteristics relating to color are referred to herein as chromatic materials. The ability of some chromatic materials to reversibly change color in response to an external stimulus such as deformation or pressure allows them to be used as a versatile pressure gauge in many applications. Materials that change color in response to deformation are mechanochromic materials, whereas materials that change color in response to pressure are piezochromic materials.

Mechanochromic materials may be comprised of flexible substances that change color when deformed. There are many variations of mechanochromic materials, but they operate on similar basic principles. As an example, polymer opals are a specific type of mechanochromic material. They are typically composed of synthetic nanospheres that are ordered in a structural sequence during the manufacturing process. The size and structure of the spheres controls how light is reflected and absorbed, which ultimately dictates how the color of the material is perceived. Deformation of the mechanochromic material changes the distance between the spheres and therefore allows light to be reflected and absorbed differently. In other words, the structural change of the nanospheres may be responsible for the change in color. This concept is further disclosed in Self-Assessing Mechanochromic Materials, U.S. Pat. No. 8,236,914 filed Jan. 26, 2010, specifically as it discusses “Color Generating Mechanophores” which is incorporated herein.

In some embodiments, the color of a chromatic material corresponds with the internal pressure of the corresponding vessel. In some embodiments, the color of a chromatic material may vary between one or more colors.

Unlike mechanochromic materials that change color due to a noticeable deformation, piezochromic materials change color in response to an applied pressure that creates structural changes on the microscopic level. These structural changes cause different wavelengths of light to be reflected or absorbed which changes how the color of the material is perceived. Piezochromic materials primarily change color in response to pressure, however, the time over which pressure is applied may also affect the change in color. Relatively high pressures may result in an immediate color change, while lower pressures may cause a color change if maintained for longer periods of time. Piezochromic materials may be applied as a coating such as a paint or powder, in some embodiments.

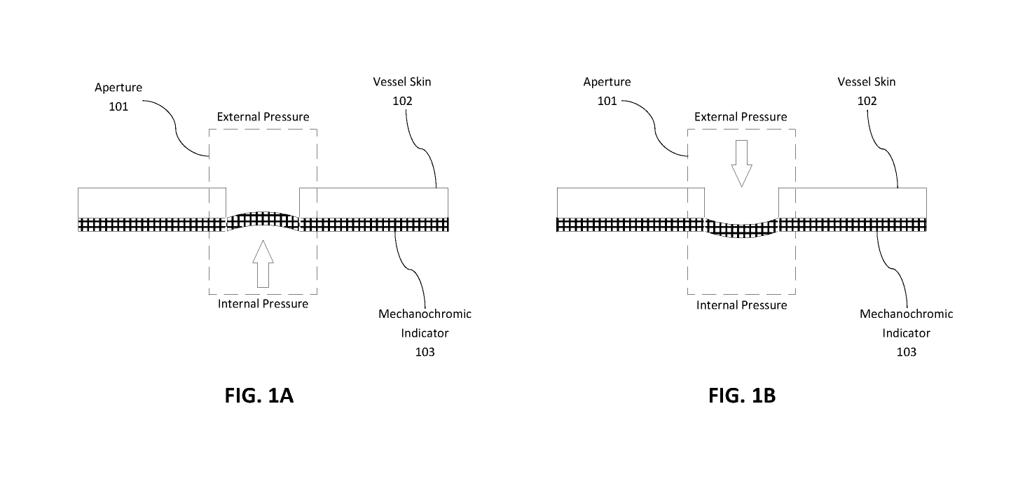

In some embodiments, as illustrated in FIG. 1A and FIG. 1B, a mechanochromic indicator 103 may be coupled to a vessel skin 102 where there is an aperture 101. A mechanochromic indicator 103 is a membrane that changes color as it deforms. In the depicted embodiment, deformation of the indicator 103 is caused by a pressure difference. The inner surface of the mechanochromic indicator 103 is exposed to the internal pressure of the vessel, and the outer surface of the mechanochromic indicator 103 is exposed to the surrounding external pressure. When the internal pressure is different than the external pressure, a pressure difference is created across the mechanochromic indicator 103. In FIG. 1A, the internal pressure is greater than the external pressure, causing the indicator 103 to expand into the aperture 101. In FIG. 1B the external pressure exceeds the internal pressure causing the indicator 103 to contract.

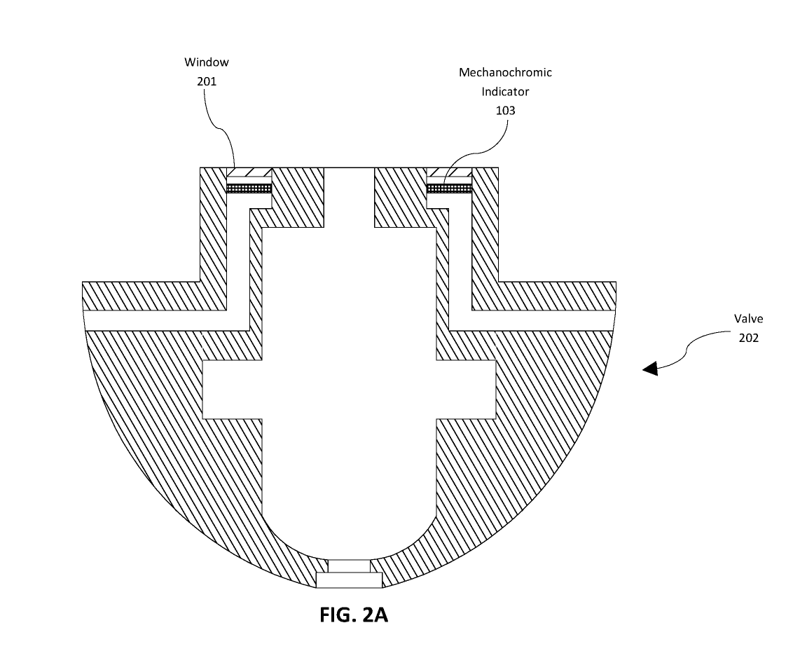

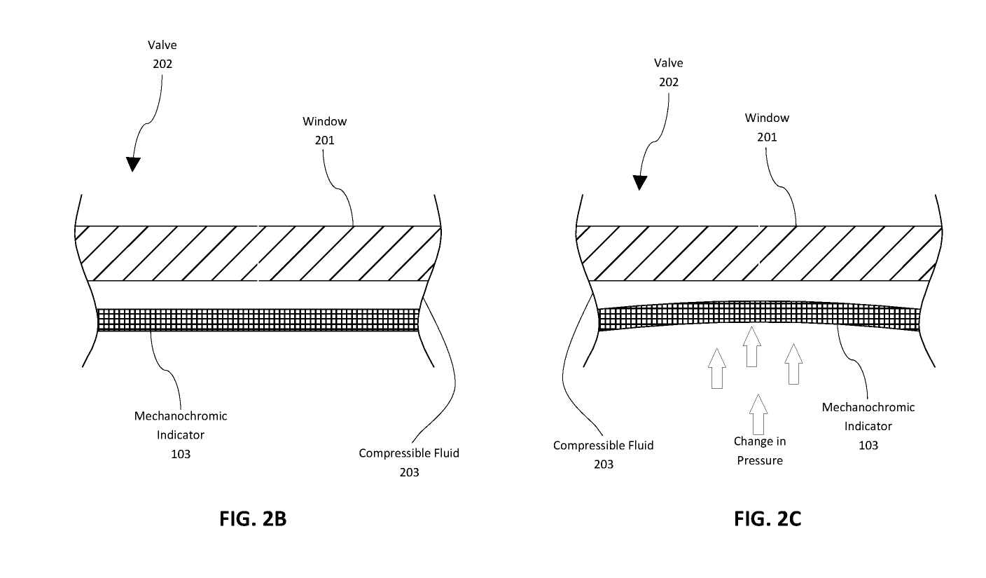

In some embodiments, as depicted in FIG. 2A, a mechanochromic indicator 103 may be integrated into a valve 202. The mechanochromic indicator 103 may be viewable through a window 201. In some embodiments, as depicted in FIG. 2B and FIG. 2C, a compressible fluid 203 may be located between the window 201 and the mechanochromic indicator 103, which may be calibrated for a predetermined pressure range. A channel may extend through the valve 202 to allow the internal pressure to reach the surface where the mechanochromic indicator 103. FIG. 2B depicts the mechanochromic indicator 103 in a non-pressurized state while FIG. 2C depicts the mechanochromic indicator 103 in a pressurized state.

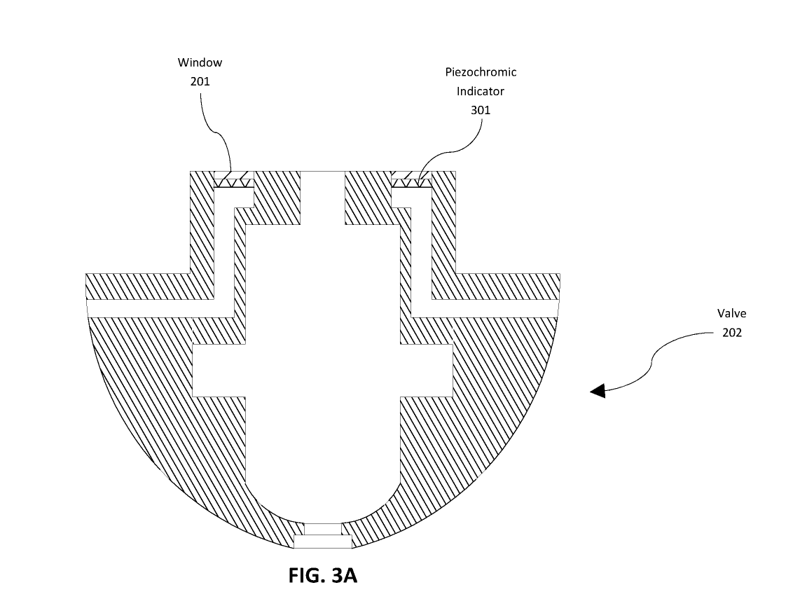

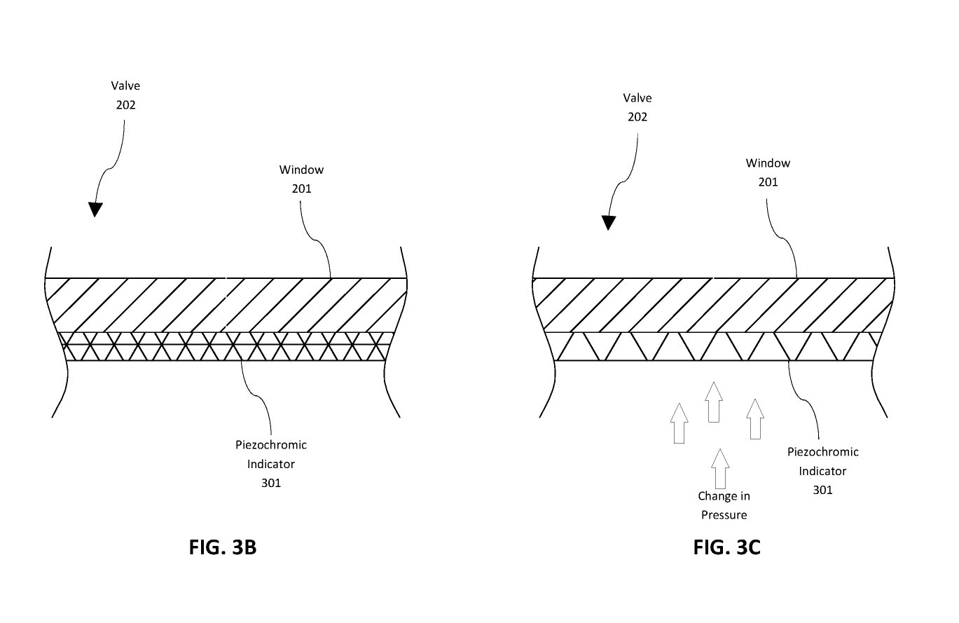

In some embodiments, as illustrated in FIGS. 3A through 3C, a piezochromic indicator 301 may be applied to a window 201 that is integrated into a valve 202. A channel may extend through the valve 202 to allow the internal pressure to act nearer to the surface where the piezochromic indicator 301 is located. The piezochromic indicator 301 may be adjacent to the outer portion of the valve 202 and have the pressure of the fluid acting on its inner surface. The piezochromic indicator 301 may be viewable through a window 201. FIG. 3B shows the piezochromic indicator 301 under a non-pressurized state. FIG. 3C depicts the piezochromic indicator 301 under a pressurized state.

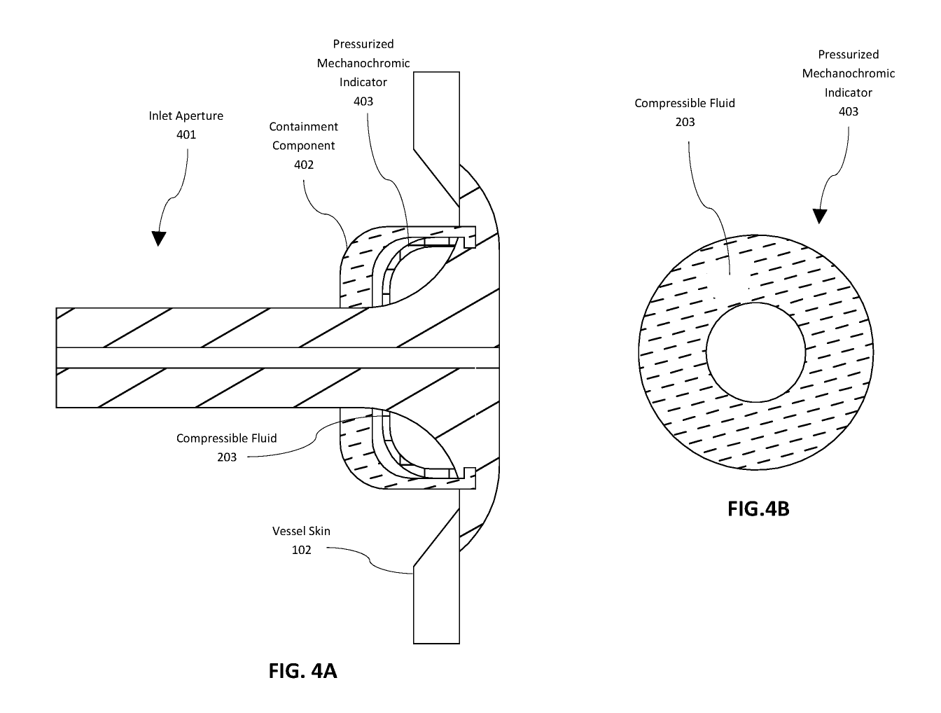

In some embodiments, as depicted in FIGS. 4A and 4B, the system includes a pressurized mechanochromic indicator 403. FIG. 4A is a cross sectional view of a valve where the exterior fluid connects to the interior fluid. FIG. 4B is a cross sectional view of an entire pressure vessel. The indicator 403 may be comprised of a malleable shell that contains a fluid. In some embodiments having a compressible fluid 203, a pressure change internal or external to the mechanochromic vessel may cause the compressible fluid 203 to vary in volume. When the properties of the compressible fluid 203 change, the properties of the pressurized mechanochromic indicator 403 are affected. The mechanochromic indicator 403 is located within a containment component 402 that surrounds an inlet aperture 401 connected to a vessel skin 102, where the vessel is to be inflated from.

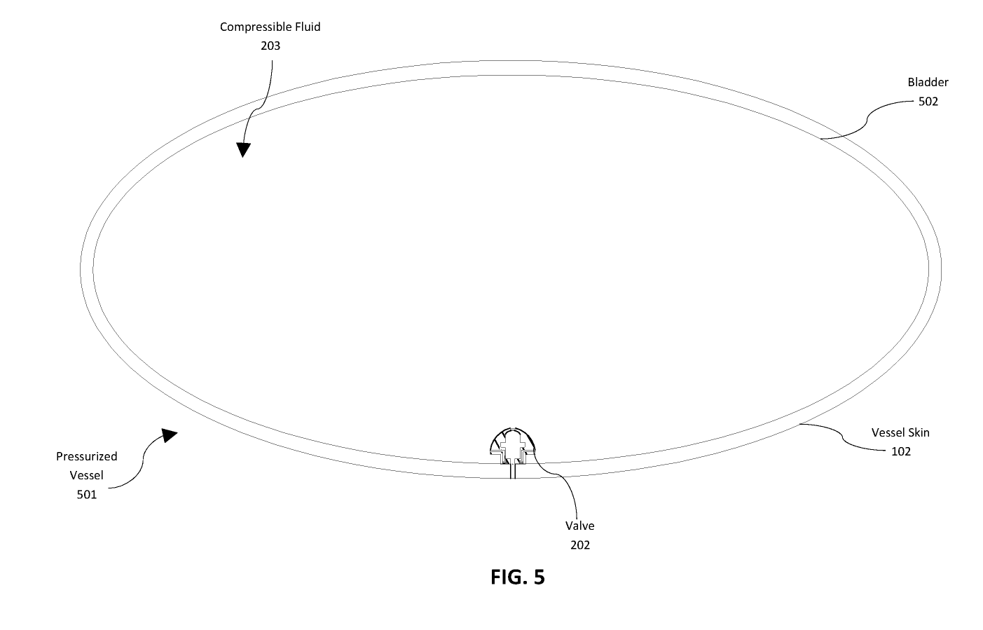

A cross sectional view of a typical pressurized vessel 501 is shown in FIG. 5. Vessels of this type are comprised of three main components: a bladder 502, a vessel skin 102, and a valve 202 that allows the bladder 502 to be filled with a compressible fluid 203. The internal pressure may be greater than, equal to, or less than surrounding pressure. The mechanochromic material may be integrated into the bladder 502, vessel skin 102, or valve 202. As the bladder 502 expands or contracts with changing internal pressure, the mechanochromic material deforms. An example of a vessel with these components and characteristics is an American football.

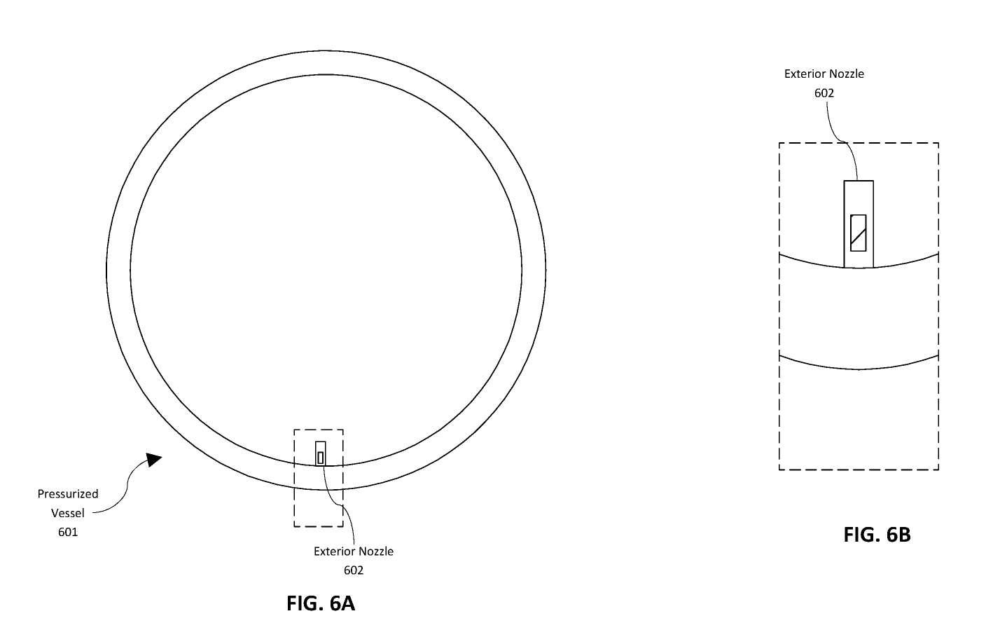

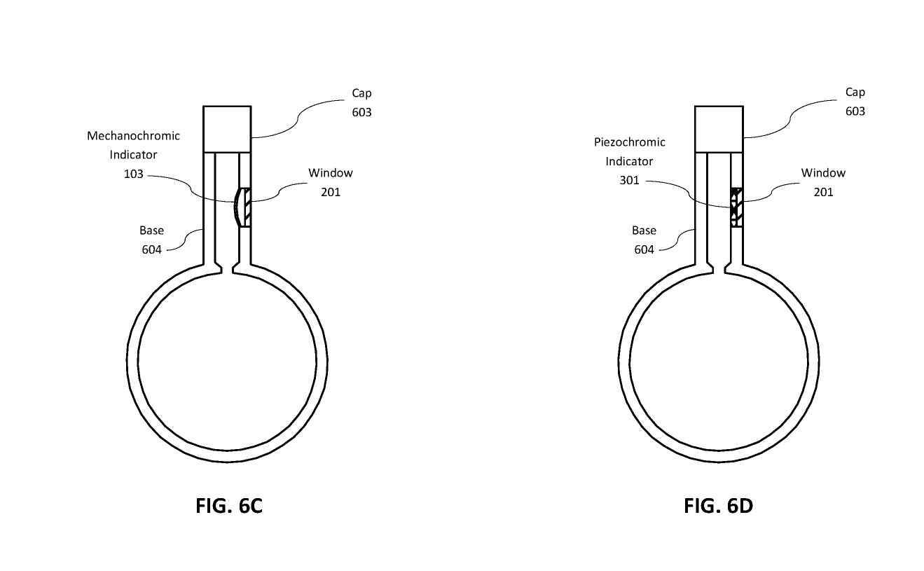

FIGS. 6A through 6D depicts a pressurized vessel in the shape of a torus. FIG. 6A is a view of the entire vessel, FIG. 6B is a close-up view of the nozzle, and FIG. 6C is a cross sectional view of the nozzle. The vessel has an exterior nozzle 602 and cap 603 and window 201. The exterior nozzle 602 may be used for adding or removing a fluid from the pressurized vessel 601. A portion of the exterior nozzle 602, commonly located below the nozzle cap 603 but above the base 604 of the nozzle 602, is subjected to the internal pressure of the pressurized vessel 601 and the surrounding pressure. A mechanochromic indicator 103 or a piezochromic indicator 301 may be incorporated into the exterior nozzle 602 to measure this pressure difference. An example of such a vessel is a bike tire valve.

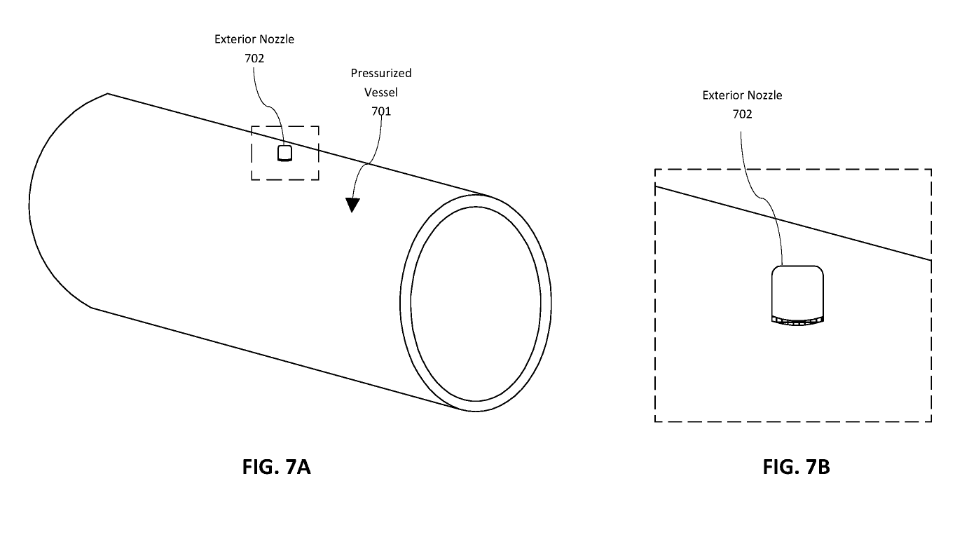

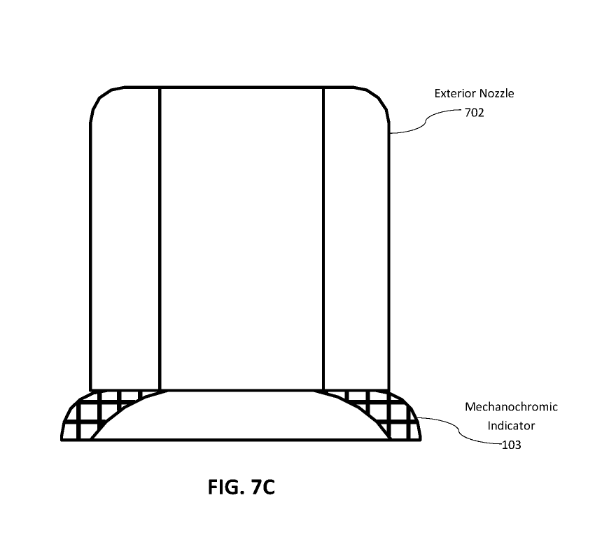

FIG. 7A illustrates a pressurized vessel 701 that is equipped with an exterior nozzle 702 such as those commonly found on portable inflatable devices. FIG. 7B shows a ring at the base of the valve that is visible under normal operating conditions. The internal surface of this ring is subjected to the internal pressure of the vessel. This portion of the exterior nozzle may be made partially or completely from a mechanochromic indicator 103. FIG. 7C is a cross sectional view of exterior nozzle 702. Swimming pool inflatables and air mattresses are examples of pressurized vessels 701 and exterior nozzles 702 of this type.

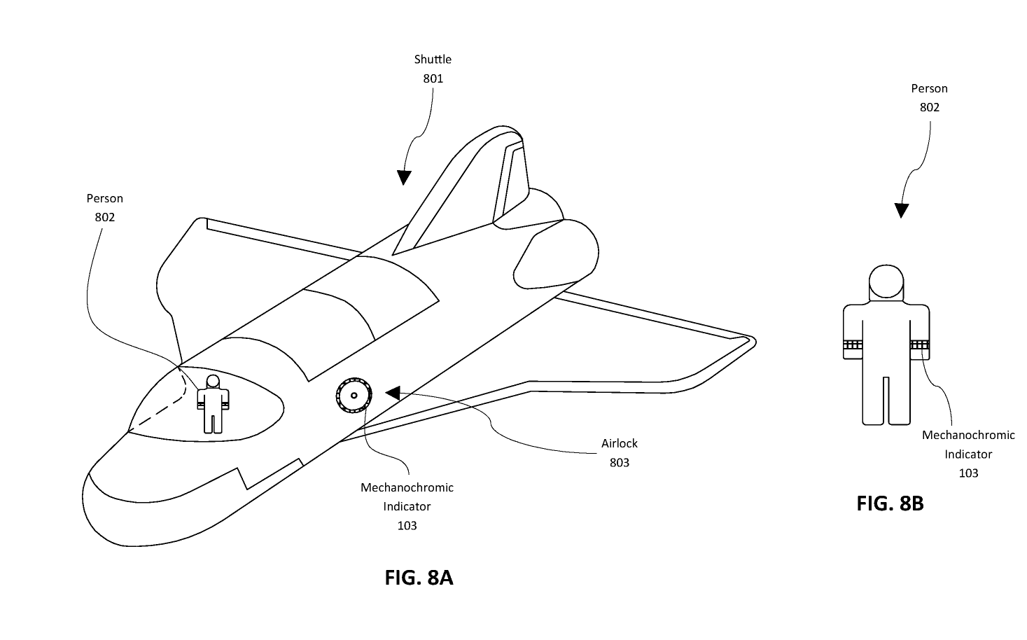

FIG. 8A depicts a space shuttle that is a pressurized vessel with airlock. In Some embodiments, a chromatic indicator 103 may also be placed in areas of oscillating pressure such as an air lock 803. In some embodiments, such as FIG. 8B, a chromatic indicator 103 may be incorporated into a space suit. The purpose is to detect pressure levels that may negatively affect the safety of the crew members 802 or the shuttle 801. In some embodiments, the pressure difference causes the chromatic material 103 to change color. The color of the chromatic material, the pressure of the space vessel 801 may be indicated via a non-electrical method. A non-electrical approach allows an independent redundant indication to add a layer of safety for the crew 802 and shuttle 801.

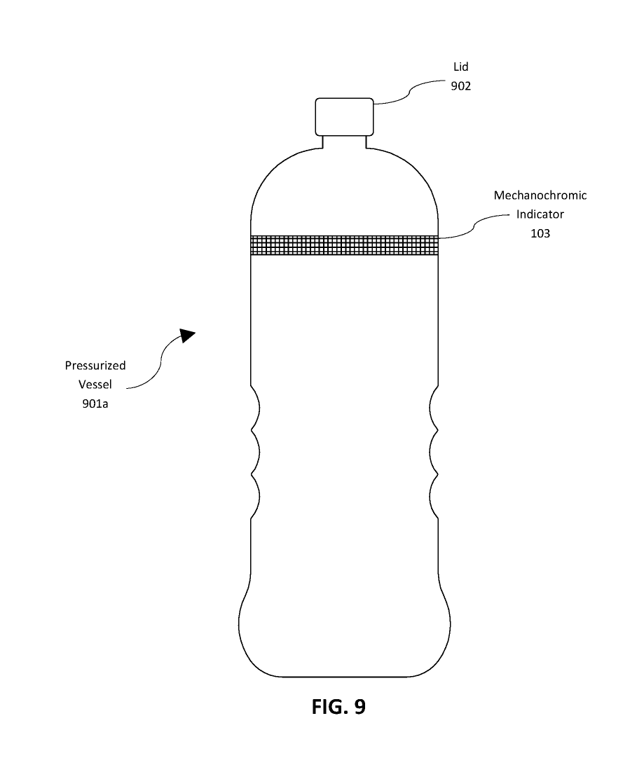

FIG. 9 depicts a pressurized vessel 901a used for carbonated beverages that may be a suitable application of a mechanochromic indicator 103. These types of pressurized vessels 901a commonly include a lid 902 to add/remove the liquid and prevent it from spilling and to maintain pressure. The pressurized vessel 901a that is depicted in FIG. 9 is a small piece of mechanochromic indicator located in the lid. The pressurized vessel can be made of a mechanochromic indicator 103 in its entirety.

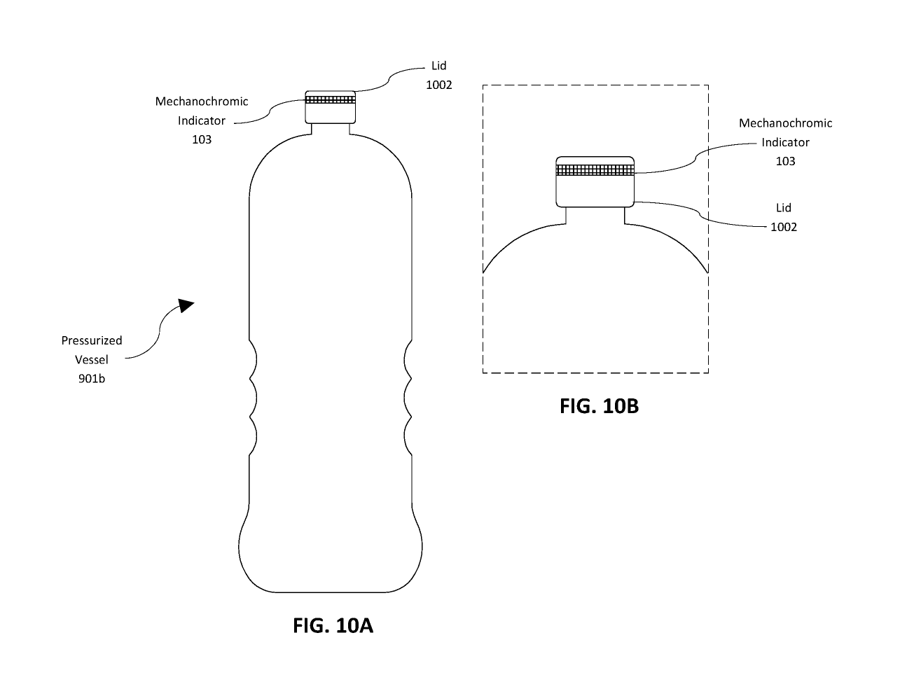

The lid 902 may also be fully or partially made of a mechanochromic material as a mechanochromic indicator 103 as shown in FIG. 10A and FIG. 10B. In some embodiments, the lid 1002 may be transparent or semi-transparent so that the inside of the pressurized vessel 901b is visible. The mechanochromic material can also be applied as a band around the outside. Benefits for manufacturers may include quality control of the pressure inside each pressurized vessel 901b. Each pressurized vessel 901b with a mechanochromic indicator 103 may be scanned with a color discerning camera, thereby each pressurized vessel 901b is assured to have a standard internal pressure. This could lower the number of pressurized vessels 901b taken from the production line for quality control testing and prevent pressurized vessels 901b that fail to meet standards from reaching the consumer. Another potential benefit of having a pressure indicator in the cap of a pressurized beverage is that the consumer can see if the beverage can be opened safely or if it has been over pressurized such as from being shaken or dropped. The mechanochromic indicator 103 may be used to alert the user that the contents have the potential to overflow if opened. The cork in a champagne bottle could also be a made of a mechanochromic indicator to serve the same purpose, as well as a band around the outside of the vessel.

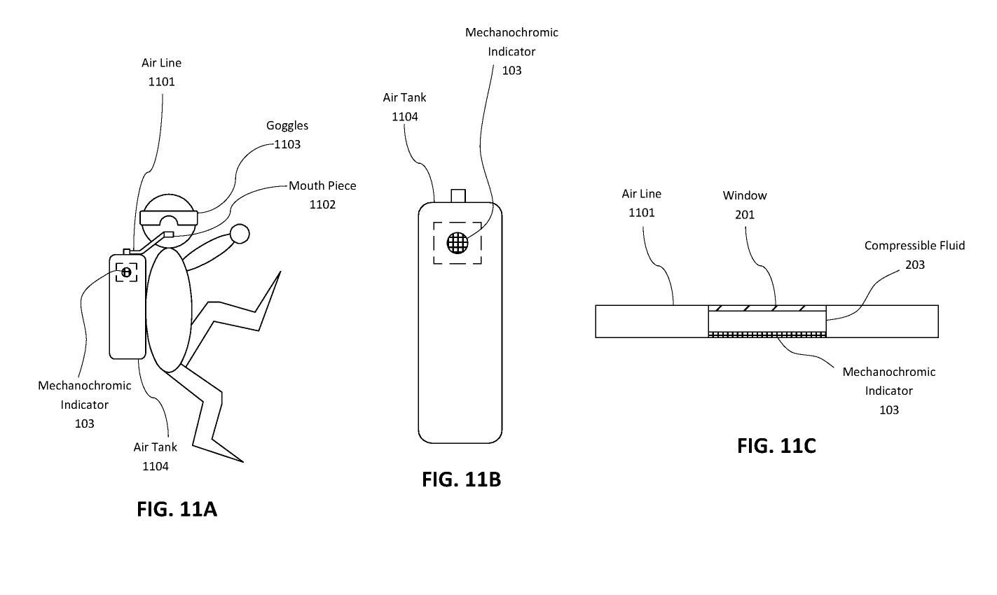

FIG. 11A depict a embodiments of mechanochromic indicators 103 being used with diving equipment. In some embodiments, the mechanochromic indicator 103 may be located about the air line 1101, goggles 1103, mouth piece 1102, or about the diving apparatus to alert the diver of dangerous pressures. For example, the goggles 1103 being pressed against the diver acts as a pressurized vessel. If the pressure difference between the water and the air inside the goggles 1103 becomes dangerous, the mechanochromic indicator 103 about the goggles 1103 will shift to a color, thus alerting the diver to increase or decrease the internal pressure of the goggles 1103.

In an embodiment illustrated in FIG. 11B a mechanochromic indicator 103 is coupled to an air tank 1104. In some embodiments, piezochromic material may be used instead of mechanochromic material.

In an embodiment illustrated in FIG. 11C a cross section of the tank is shown to display the mechanochromic indicator on the interior side of the tank and a window on the exterior side of the tank.

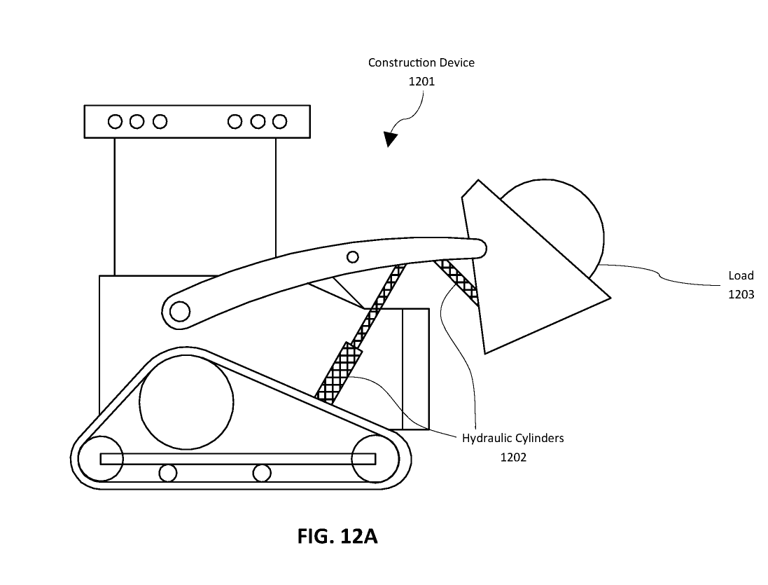

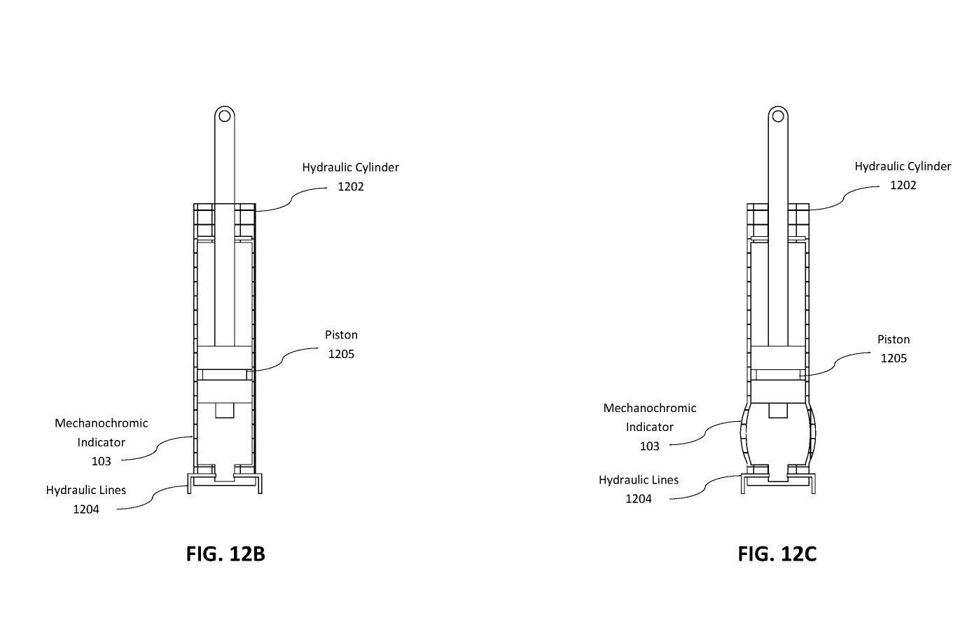

In some embodiments, as illustrated in FIGS. 12A through 12C, piezochromic or mechanochromic indicators can be employed into a hydraulic cylinder 1202 for mechanical failure analysis. Hydraulic cylinders 1202 are primarily used to supply a unidirectional force and can often be seen in a construction device 1201 to aid with the lifting of loads 1203. Piezochromic material can be applied as a Mechanochromic indicator 103 within the inner surface of the hydraulic cylinder 1202 barrel to indicate the pressure of the hydraulic fluid. In some embodiments, a window may be fitted on the side of the hydraulic cylinders 1202 walls to view the piezochromic color change. The color change can be calibrated to correspond to a known fluid pressure. Mechanochromic material can also be applied along the outer surface of the hydraulic cylinder 1202. The Mechanochromic material can act as a visual strain gauge, indicating a significant deformation to the hydraulic cylinder 1202 and possible failure of the hydraulic actuator.

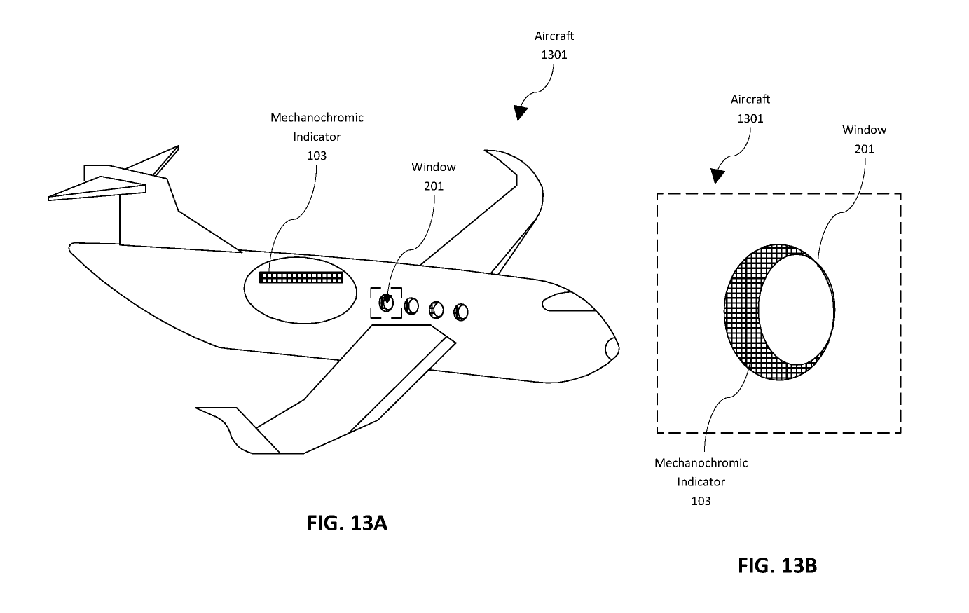

FIG. 13A depicts a mechanochromic indicator 103 used on an aircraft 1301. In some embodiments, the indicator can be applied on the windows 201 and within the cabin of the aircraft 1301. FIG. 13B depicts the application of the mechanochromic indicator 103 fitted on the outside of an aircraft 1301 window 201. In some embodiments, a mechanochromic indicators 103 is attached within the cabin of the aircraft 1301, via a reinforced strip to maintain the structural integrity of the aircraft 1301. These embodiments would allow the pressure inside and outside of the aircraft 1301 to be observed from the inside the aircraft 1301.

It should be clear that while many embodiments are discussed as separate wholes from other embodiments that various aspects from any one or more embodiments may be combined to form other embodiments not explicitly disclosed herein.

For the sake of convenience, the operations are described as various interconnected functional blocks or distinct software modules. This is not necessary, however, and there may be cases where these functional blocks or modules are equivalently aggregated into a single logic device, program, or operation with unclear boundaries. In any event, the functional blocks and software modules or described features can be implemented by themselves, or in combination with other operations in either hardware or software.

Having described and illustrated the principles of the systems, methods, processes, and/or apparatuses disclosed herein in a preferred embodiment thereof, it should be apparent that the systems, methods, processes, and/or apparatuses may be modified in arrangement and detail without departing from such principles.

1. A system for determining pre-set pressure parameters of a vessel, comprising:
a boundary that contains one or more fluids;
one or more vessel apertures, wherein the one or more vessel apertures may allow fluid to at least one of enter and exit the vessel;
one or more chromatic materials, wherein the one or more chromatic materials change color dependent on at least one of applied pressure and deformation and wherein the color change is reversible.

2. The system of claim 1, wherein the one or more chromatic materials comprise a piezochromic material.

3. The system of claim 1, wherein the one or more chromatic materials comprise a mechanochromic material, wherein color change is dependent upon deformation, wherein deformation is dependent upon pressure.

4. The system of claim 1, wherein the one or more vessel apertures comprise a valve comprising a mechanochromic membrane, a transparent window, and a fluid between the window and membrane.

5. The system of claim 1, wherein the vessel is a piston-cylinder and the outer surface of the vessel is made of one or more mechanochromic materials.

6. The system of claim 1, wherein the one or more vessel apertures comprise a valve comprising a transparent window on the surface, and a chromatic paint, wherein the chromatic paint is applied on the inside surface of the transparent window.

7. The system of claim 1, wherein an overall vessel comprises of a subsequent vessel made of one or more chromatic material and that contains a fluid, wherein the subsequent mechanochromic vessel may deform and change color with the pressure of the overall vessel.

8. The system of claim 1, wherein the vessel further comprises a bladder and a vessel skin.

9. The system of claim 1, wherein the vessel is in the shape of a torus and an inlet aperture is an exterior nozzle that extends outward from a vessel surface.

10. The system of claim 1, wherein the one or more vessel apertures comprises an exterior nozzle that extends outward from a surface of the vessel and one or more chromatic materials are located inside a valve stem under a transparent section of the valve stem.

11. The system of claim 1, wherein the one or more vessel apertures comprises an input nozzle that comprises one or more chromatic indicator where it couples to the vessel.

12. The system of claim 1 wherein the vessel is a pressurized suit and contains a person and comprises an aperture and chromatic material allowing the pressure of the vessel to be analyzed by the person within.

13. The system of claim 1, wherein the aperture is an opening and wherein the aperture further comprises of a lid and one or more chromatic indicators.
